(19) United States
(12) Patent Application Publication
(10) Pub. No.: US 2022/0233325 A1
(43) Pub. Date: Jul. 28, 2022

(54) WRIST ENDOPROSTHESIS

(71) Applicant: Rheinisch-Westfälische Technische Hochschule Aachen (RWTH), Aachen (DE)

(72) Inventors: Sophia LEVEN, Aachen (DE); Jörg ESCHWEILER, Heimbach-Vlatten (DE); Markus TINGART, Aachen (DE); Björn RATH, KRENGLBACH (AT)

(73) Assignee: Rheinisch-Westfälische Technische Hochschule Aachen (RWTH), Aachen (DE)

(21) Appl. No.: 17/614,448

(22) PCT Filed: May 26, 2020
(86) PCT No.: PCT/EP2020/064595

§ 371 Date: Nov. 26, 2021

(30) Foreign Application Priority Data
May 28, 2019 (DE) 10 2019 114 314.4

(51) Int. Cl. A61F 2/42

(52) U.S. Cl. CPC A61F 2/4261; A61F 2002/3082; A61F 2002/4287; A61F 2002/4264

(57) ABSTRACT

A wrist endoprosthesis (2) for functional replacement of the human wrist, containing a radius component (4) that has a shaft (10) for anchoring in the radius, a head (12), and a first joint surface (16), which is implemented on a distal head face (14), and a carpal component (6) that has a proximal carpal face (22), a distal carpal face (20) and a second joint surface (24) which is formed on the proximal carpal face (22) and interacts with the first joint surface (169) of the radius component (4), characterized in that the carpal component (6) is substantially trough-shaped, in order to at least partially surround the carpal bones. Also, a wrist endoprosthesis (2) that has anti-luxation protection (8), a method for producing wrist endoprostheses (2) and a computer program product.

The invention relates to a wrist endoprosthesis comprising a radius component that has a shaft for anchoring in the radius, a head, and a first joint surface, which is formed on a distal head face; and comprising a carpal component that has a proximal carpal face, a distal carpal face and a second joint surface, which is formed on the proximal carpal face and interacts with the first joint surface of the radius component. The invention also relates to a method for producing a wrist endoprosthesis of the type named above and a computer program product.

Total wrist endoprostheses are used for treating various diseases, such as rheumatoid arthritis, or damaging traumatic injuries to the wrist. For example, the product Remotion from the manufacturer Stryker Corporation or WO 2008/024853 A2 are known as disclosed wrist endoprostheses. These are total wrist endoprostheses that have a radius component, which is anchored with a shaft in the radius of a patient, and a carpal component. Both the radius component and the carpal component each have a joint surface which interacts with the joint surface of the other component and assumes the joint function of the natural wrist of the patient. What is common to both wrist endoprostheses is that the carpal component is anchored in the interior of the carpal bones of a patient with the aid of screws and pins. For this purpose, the bones of the first carpal row are completely or partially removed and the bone marrow spaces in the region of the anchoring pins and screws are opened up to a large extent. The resulting serious invasive trauma complicates subsequent treatment and is the cause of a lengthy healing process. In addition, in the event of a prosthesis failure, under some circumstances, there may not be enough bone material available for a new prosthesis restoration.

Wrist endoprosthetics are still a relatively unexplored domain, especially when compared to hip and knee arthroplasty. The complexity of the movements in the wrist makes it much more difficult to imitate the physiological movements with an endoprosthesis, and attaching the carpal component is difficult due to the small size of the carpal bones. Known wrist endoprostheses often have similar failure patterns and a limited service life of the prosthesis. The most common failure patterns include loosening and fractures of the prosthesis and luxations. Loosening can occur on the carpal component and on the radius component. There are many causes of a loosening of the prosthesis. In addition to infections and technical defects during implantation (for example, incorrect positioning/orientation), the different mechanical behaviour between implant and bone, the complex movement patterns and the small attachment surface play a decisive role. In addition, a rigid implant absorbs the forces acting on the bone, which can lead to remodelling processes or to osteoporosis and thus to loosening of the implant. Said effects are reinforced by the use of standardized prostheses, since the predefined size relationships thereof are often not compatible with the real geometric relationships in the wrist of a patient. An additional factor that often occurs in connection with negative aspects of wrist endoprosthesis care is the restricted range of motion due to a lack of conformity of the joint surfaces of the wrist endoprosthesis with the original natural joint surfaces of the patient to be treated. Due to the diverse failure patterns, the service life of wrist endoprostheses, as currently in the prior art, is often severely limited.

Further wrist endoprostheses are known from WO 2018/086765 A1 and WO 2018/053431 A1. The known wrist endoprostheses are partial prostheses which are not suitable for the treatment of serious injuries and/or diseases of the entire human wrist apparatus. Furthermore, a production method for prostheses is known from US 2017/0360578 A1 and a finger prosthesis is known from US 2002/0082705 A1.

The present invention is therefore based on the object of providing a wrist endoprosthesis that has an extended service life.

In a first consideration of the invention, the stated object is achieved with a wrist endoprosthesis of the type named above in that the carpal component is formed substantially trough-shaped in order to at least partially surround the carpal bones.

The invention is based on the knowledge that a trough-shaped carpal component enables a particularly simple and secure fixation of the carpal component on the carpal bones of a patient. In addition, bone resections can be minimized or ideally avoided entirely when using a trough-shaped carpal component. The carpal component is that component of a wrist endoprosthesis according to the invention which is formed for attachment to the carpal bones of a patient. A trough shape allows the carpal component to be at least partially pushed over one or more carpal bones of a patient to be treated, so that the carpal bones are at least partially disposed in the interior of the carpal component. If the one or more carpal bones are disposed in the interior of the carpal component, said carpal bones are surrounded on at least three faces by the carpal component. The carpal bones are preferably surrounded on a palmar, dorsal and proximal carpal face by the trough-shaped carpal component. A wrist endoprosthesis implemented in this way can achieve a longer service life compared to the prior art.

A proximal carpal face is that face of the carpal component which, when used according to the invention, points in the direction of the centre of the body of a patient. The second joint surface is formed on said proximal carpal face. A palmar carpal face is that face of the carpal component which, when used according to the invention, points in the direction of the palm of a patient. The palmar carpal face is substantially perpendicular to the proximal carpal face. The dorsal carpal face is opposite the palmar carpal face. The distal face of the carpal bones is that face which is further away from the centre of the body. In an analogous manner, a face of the carpal component opposite the proximal carpal face is referred to as the distal carpal face.

A trough-shaped carpal component surrounding the one or more carpal bones allows the carpal bones to be integrally cast into the carpal component or the carpal bones to be glued to the carpal component. This enables secure and reversible attachment of the carpal component to the carpal bones of a patient. The carpal bones are preferably surrounded by bone cement or some other medical potting agent, wherein a loosening of the carpal component is avoided. There is no need to attach the carpal component with the aid of screws and pins that extend into the carpal bones from a proximal face in the direction of a distal face. Bone resections can also be avoided or minimized. Furthermore, the carpal component can preferably comprise screws or pins extending from the palmar carpal face to the opposite dorsal carpal face and are provided for further fixing the carpal component in the region of the carpal bones. Also preferred are screws or pins which extend from the dorsal carpal face to the palmar carpal face of the carpal component.

The trough-shaped carpal component is preferably elongated, wherein an extension in a first direction, measured between the palmar carpal face and the dorsal carpal face, is less than an extension in a second direction perpendicular to said first direction. In a particularly preferred embodiment, a thickness of the head of the radius component, measured between the first joint surface and a connecting segment to the shaft, on a radius face of the head is greater than a corresponding thickness of the head on an ulnar face of the head. The first joint surface is preferably implemented to be substantially concave in a sagittal plane and/or a frontal plane. The frontal plane corresponds to a plane that is substantially parallel to the palmar and dorsal carpal faces.

In a first preferred embodiment, the trough comprises a carpal cavity which is open to the distal carpal face and forms the trough shape. Said carpal cavity is used for receiving the one or more carpal bones of a patient. The carpal component particularly preferably comprises an inner surface which is formed by the carpal cavity. It is further preferred that the inner surface comprises a surface structure which is implemented for improved adhesion of bone cement. For this purpose, the surface structure particularly preferably comprises an increased roughness and/or small elevations. In a further preferred embodiment, the inner surface is substantially continuous. A continuous inner surface that is free of edges and/or projections can avoid unpleasant or harmful loads on the bone structure of a patient. This can extend the service life.

In a further preferred embodiment, the carpal cavity is substantially U-shaped in a sagittal plane. As a result, the at least one carpal bone can be surrounded by the carpal cavity both on the palmar face and on a dorsal face. The sagittal plane is disposed substantially perpendicular to the palmar and the proximal carpal face and is preferably a sectional plane of the carpal component. A contact surface between the carpal cavity and the at least one carpal bone is enlarged. If the carpal component is fixed by means of a potting agent, for example, bone cement, the strength of the fixation can thus be improved. Due to the U-shaped carpal cavity, impairments and punctual loads on the at least one carpal bone can be avoided and a fit of the carpal cavity can be improved. Furthermore, the U-shaped structure ensures increased mechanical stability of the carpal component and an even load distribution. In addition, the U-shaped structure of the carpal cavity enables improved filling with potting agent. For example, bone cement can slowly penetrate into spaces between the bones and/or the formation of cavities can be avoided.

The carpal cavity preferably has a maximum clear width, measured in the sagittal plane, wherein a corresponding clear width of the carpal cavity at a distal end of the carpal component is smaller than the maximum clear width. The distal end of the carpal component is located on the distal carpal face and, when used according to the invention, points in the direction of the fingertips of a patient. The inner cavity therefore preferably comprises an undercut in the sagittal plane and/or tapers in the direction of the distal end. Such a shape of the inner cavity can achieve a positive and/or material connection of the carpal component with the one or more carpal bones of the patient after the at least one carpal bone has been socketed in the interior of the cavity. Slipping out of the at least one carpal bone in connection with the potting agent can be avoided. It is further preferred that the maximum clear width of the inner cavity is disposed between a proximal end and a distal end of the carpal cavity.

In a particularly preferred embodiment, the carpal component comprises one or two lateral openings which are implemented in a face parallel to the sagittal plane. The face of the carpal component parallel to the sagittal plane is preferably an ulnar face or radial face of the carpal component. The ulnar face is substantially perpendicular to the proximal, distal, dorsal and palmar carpal face of the carpal component and, when used according to the invention, points in the direction of the ulna of a patient. The one or two lateral openings can facilitate attaching of the carpal component and reduce the weight thereof. The lateral openings can extend entirely or partially over the ulnar face and/or radial face.

The one or two lateral openings are particularly preferably implemented as substantially Us shaped slots that are open towards the distal face. The slots opened in the direction of the distal carpal face enable the carpal component to partially surround one or more carpal bones in a direction between the distal and proximal carpal face. For example, the navicular bone, lunar bone, triangular bone and small polygonal bone of a patient can be disposed completely in the carpal cavity, while the large polygonal bone, the head bone and/or the hook bone are only partially disposed within the carpal cavity and extend partially or completely through the slots. For example, the head bone and the hook bone can also extend through the slots, while the large polygonal bone lies entirely outside the cavity. In order to avoid injuries to the carpal bones, the edges of the openings are preferably rounded. It is also preferred that edges of the carpal cavity, which form a transition between the carpal cavity and one or more outer surfaces of the carpal components, are rounded.

In a further preferred embodiment, the carpal cavity comprises a concave bulge which is implemented in the direction of the proximal carpal face. The bulge increases the volume of the carpal cavity and forms a concave bottom of the carpal cavity. Due to the bulge, the carpal cavity has a maximum depth, measured in an axial direction between the distal and proximal carpal faces. The maximum depth is greater than the corresponding depths of the carpal cavity on the ulnar face and the radial face. A depth of the lateral openings, measured in the axial direction, is particularly preferably less than the maximum depth of the carpal cavity. The concave bulge preferably corresponds substantially to a negative shape of the proximal face of one or more carpal bones of a human wrist. The concave bulge makes it easier to receive the navicular and/or lunar bone of a patient in the carpal cavity, and the occurrence of punctual loads on the carpal bones can be avoided.

The carpal component preferably tapers parallel to the frontal plane in the direction of the distal carpal face. A first width of the carpal component, measured between the ulnar face and the radial face, is therefore preferably less on the distal carpal face than a corresponding second width of the carpal component in a central region which is disposed between the proximal carpal face and the distal carpal face. The carpal component preferably tapers in a substantially trapezoidal shape parallel to the frontal plane in the direction of the distal carpal face, particularly preferably tapering in a substantially isosceles trapezoidal shape. Impairment of the freedom of movement of the fingers of a patient can be minimized as a result of the carpal component tapering in the direction of the distal carpal face.

In a further preferred embodiment, a proximal end of the carpal component is implemented as a thickening. The preferred thickening can increase the mechanical stability of the carpal component at the proximal end. In addition, pressure forces that act on the second joint surface can be introduced evenly into the carpal bones. A first wall thickness at the distal end of the carpal component is preferably less than a second wall thickness at the proximal end. Furthermore, the proximal end is preferably substantially convex in the frontal plane and/or in the sagittal plane. The second joint surface is particularly preferably disposed at least partially on the thickening.

The carpal component is particularly preferably substantially mirror-symmetrical with respect to a frontal plane. In a further preferred embodiment, the carpal component is mirror-symmetrical to a sagittal plane. A mirror-symmetrical design of the carpal component can facilitate production both by means of machining manufacturing processes and by means of additive manufacturing processes. For example, the creation of a model, production of the carpal components by means of 3D printing and/or post-processing of the carpal components can be improved. Since twisting or incorrect insertion of the carpal component is prevented by the symmetrical shape, the reliability of the implantation can also be increased. Furthermore, a symmetrical shape ensures that the joint surfaces slide smoothly.

In a second consideration of the invention or a preferred refinement, the object named above is achieved in that the wrist endoprosthesis comprises luxation protection. The occurrence of complete or incomplete loss of contact between the carpal component and the radial component can be reduced by the luxation protection, whereby damage to the prosthesis or the surrounding tissue can be avoided. As a result, the service life of the wrist endoprosthesis can be increased by the luxation protection. The luxation protection is preferably implemented as a mechanical component which restricts the freedom of movement of the carpal component relative to the radius component. Movements that do not change a distance between the joint surfaces, as is the case, for example, for rotational and pivoting movements, are particularly preferably not restricted. It is further preferred that the luxation protection connects the carpal component and the radius component.

In a particularly preferred embodiment, the luxation protection comprises a band that connects the carpal component and the radius component. The band is preferably formed from a plastic, a fibre material and/or a metallic material. Likewise, luxation protection which comprises a plurality of bands connecting the carpal component and the radius components is also preferred. It is further preferred that a tension force is applied by the band, which clamping force brings the first and second joint surfaces into contact and/or counteracts a lifting of the carpal component from the radius component. The clamping force can preferably be individually adapted to the patient before and/or during the implantation.

The radius component preferably comprises a tunnel which extends from a dorsal face of the radius component to a palmar face of the radius component, wherein the band runs through the tunnel. The tunnel preferably runs between two faces of the head. The tunnel particularly preferably runs from a first face of the head, which first face corresponds to the dorsal face of the radius components, to a second face of the head, which second face corresponds to the palmar face of the radius component. It should be understood that the tunnel in the interior of the radius component can also extend into the shaft. The head of the radius component is provided to protrude from a distal end of the radius after implantation of the radius component in the radius of a patient, while the shaft is anchored in the radius. The palmar face of the radius component and the palmar carpal face are disposed on the same face of the wrist endoprosthesis. The dorsal face of the radius component is an opposite face from the palmar face. A first and a second access to the tunnel are accessible after the implantation by extending the tunnel between the faces of the head. The band running through the tunnel can preferably execute a relative movement through the tunnel. The tunnel particularly preferably comprises an oval, round or slot-shaped cross section, particularly with appropriately adapted openings. This can minimize band friction. It is further preferred that the tunnel extends from an ulnar face to a radius face of the head. It is also preferred that a second tunnel extends from the ulnar face to a radius face of the radius component and a second band of the luxation protection extends through the second tunnel. In addition, embodiments are preferred that have a plurality of tunnels which extend from the ulnar face to a radius face of the radius component, wherein further bands of the luxation protection run through the tunnels. It can also be provided that a width of the band substantially corresponds to a width of the tunnel, so that twisting of the band is avoided.

In a further preferred embodiment, the tunnel is curved in the direction of a proximal end of the shaft. The proximal end of the shaft lies opposite the distal head face and, when used according to the invention, is the segment of the wrist endoprosthesis that is closest to the centre of the body of a patient. The tunnel is particularly preferably curved in the direction of the shaft such that a maximum distance of the tunnel from the first joint surface lies on a central axis of the radius component, which runs from the proximal end of the shaft to the distal head face. Curving the tunnel can minimize friction load on the band. Openings in the tunnel are preferably rounded for this reason.

The band particularly preferably comprises a first end connected to a palmar carpal face and a second end connected to a dorsal carpal face. A first length of the first end and a second length of the second end are preferably variable due to the relative movement of the band through the tunnel. The connection of the first end to the palmar face and/or the connection of the second end to the dorsal face is preferably material, frictional and/or positive. A connection of the first and/or second end on a shoulder of the carpal component, which forms a transition region to the thickening, is preferred.

In a particularly preferred embodiment, a first material forming the wrist endoprosthesis is an isoelastic material, preferably a thermoplastic, particularly preferably polyetheretherketone (PEEK). An isoelastic material has one or more mechanical properties that substantially correspond to the mechanical properties of a human bone in the region of the wrist. Particularly preferably, a modulus of elasticity of the first material substantially corresponds to the modulus of elasticity of a human radius. Segments of the wrist endoprosthesis formed from an isoelastic material show a reaction to acting forces which substantially corresponds to the reaction of a real bone. Compared to rigid implants that absorb the forces acting thereon, remodelling processes in the bone structure surrounding the prosthesis and the resulting loosening can be avoided. In addition, harmful relative movements between the wrist endoprosthesis and the bones attached thereto are prevented. PEEK has such isoelastic properties and is also biocompatible. The use of other supplementary materials such as titanium is further preferred. These can be used, for example, for increasing mechanical stability or bone affinity. The further material is particularly preferably added to the isoelastic material and/or the isoelastic material is completely or partially coated with the further material.

In a third consideration, the invention solves the problem named above with a wrist endoprosthesis for partial functional replacement of the human wrist, comprising: a carpal component that has a proximal carpal face, a distal carpal face and a second joint surface, which is disposed on the proximal carpal face and is formed for interaction with a distal joint surface of a human radius, characterized in that the carpal component is substantially trough-shaped, in order to at least partially surround the carpal bones. If a bony structure of the radius is still largely present on the wrist to be treated, the carpal component can also interact directly with the joint surface of the human radius that is still present. The wrist endoprosthesis can then also be designed without a radius component and/or only comprise a carpal component. Fewer steps are necessary and the patient can be spared when the wrist endoprosthesis is implanted. Owing to the trough shape of the carpal component particularly, bone resections, that is, the removal of bone material, can also be completely dispensed with. The carpal component surrounds the carpal bones and interacts with the second joint surface with the distal joint surface of the human radius. The wrist endoprosthesis then partially assumes the function of the human wrist. The wrist endoprosthesis can preferably comprise luxation protection, which is implemented to be fixed to a human radius component.

In a first preferred refinement, the proximal carpal face comprises a thickening that is implemented as a convex elevation. A convex elevation is particularly adapted to the anatomy of the distal joint surface of a human radius. Furthermore, the thickening allows a dimensionally stable structure. Irritation and/or wear and tear of the radius of a patient interacting with the carpal component are thus effectively prevented.

In a fourth consideration of the invention, the object named above is achieved by a method for producing a wrist endoprosthesis, preferably according to one of the preferred embodiments of a wrist endoprosthesis described above, wherein the method comprises the steps: providing or producing a non-individualized 3D model of a wrist endoprosthesis; acquiring data material, particularly x-ray images, magnetic resonance tomography data, ultrasound images and/or computed tomography data, of a wrist of a patient to be treated; determining one or more parameters from the data material for approximating an approximated joint structure of the patient from the data material; individualizing the non-individualized 3D model based on the derived parameters to obtain an individualized 3D model; and producing the wrist endoprosthesis by means of an additive manufacturing process based on the individualized 3D model. With the method, wrist endoprostheses can be produced which are individually adapted and custom-made to the geometry of the wrist of a patient to be treated. An improved implant fit or integration into the joint chain, lower postoperative pain, improved postoperative function and/or lower operative blood loss can thereby be achieved. Protrusions and the resulting soft tissue injuries can be avoided by using individualized prostheses. Production by means of an additive manufacturing process, such as a 3D printing process particularly, enables production costs, material consumption and/or the production time to be reduced. In addition, very complex geometric shapes can be produced. The 3D printing process Fused Deposition Modelling (FDM) is particularly preferred. In FDM, a filament, preferably a plastic filament, particularly preferably PEEK, is melted and applied via a mechanism, wherein the object to be created is built up in layers. The production and individualization of the 3D model can be carried out based on CAD applications. One or more of the steps mentioned are preferably carried out in an automated manner. Preferably, only subcomponents of the wrist endoprosthesis can be produced by means of an additive manufacturing process. Furthermore, the wrist endoprosthesis and/or subcomponents of the wrist endoprosthesis can preferably also be produced by means of a machining manufacturing process based on the individualized 3D model.

In a first preferred refinement, the parameter represents at least one geometric shape or form, preferably full shape, of a ligamentous connection between adjacent carpal bones of the proximal carpal row of the wrist to be treated, and the individualized 3D model comprises an elevation corresponding to the recess on a face facing the anatomy. The anatomy-facing face is a face of the 3D model or of the wrist endoprosthesis to be produced which faces the anatomy, particularly the bony structure, of the wrist to be treated. If the wrist endoprosthesis comprises a trough-shaped carpal component according to the first consideration of the invention, the face facing the anatomy is defined by the carpal cavity. The ligamentous or band-adhering connections between the carpal bones are characterized by recesses which, in a healthy wrist, are filled with soft tissue. An elevation corresponding to at least one of said recesses on the anatomy-facing face of the wrist endoprosthesis can be placed on said recess and prevents the prosthesis from slipping after implantation. Furthermore, the risk of incorrect positioning is reduced. The face facing the anatomy is preferably a segment-wise geometric negative of the bony structure of the wrist to be treated.

The method preferably further comprises: providing a surface structuring on at least one surface segment of the anatomy-facing face of the individualized 3D model, wherein the production of the wrist endoprosthesis takes place based on the individualized 3D model with surface structuring. The surface segment of the face facing the anatomy is preferably smooth before the surface structuring is provided. The surface structuring is preferably provided on the entire anatomy-facing face. The surface structuring is also present on the anatomy-facing face of the produced wrist endoprosthesis, since said wrist endoprosthesis is produced based on the 3D model with surface structuring. The surface structuring then brings about a better and/or more secure fit of the wrist endoprosthesis, since the total surface area of the face facing the anatomy is increased. The surface structuring is preferably generated randomly.

In a preferred embodiment, a second parameter represents a shape and position of a cartilage-covered joint surface of the wrist to be treated and a third parameter represents a shape and position of a cartilage-free surface of the wrist to be treated, wherein the individualized 3D model comprises a first connecting surface corresponding to the cartilage-covered joint surface and a second connecting surface corresponding to the cartilage-free surface on the face facing the anatomy. Cartilage-covered joint surfaces typically move relative to other bones as the wrist moves and are covered with cartilage to protect against wear. Proximal segments of the proximal carpal row are particularly covered with cartilage at least in segments. Distal segments of the proximal carpal row are usually not or not completely covered with cartilage and are therefore free of cartilage. By providing corresponding first and second connecting surfaces, the wrist endoprosthesis can advantageously be adapted to mutually different properties of the cartilage-covered joint surface and the cartilage-free surface of the wrist to be treated. The position of the connecting surfaces corresponds to the respective surfaces of the wrist. If the finished wrist endoprosthesis is inserted into the wrist to be treated, the first connecting surface rests against the cartilage-covered joint surface or is disposed adjacent thereto. The second connecting surface is then associated with the cartilage-free surface in an analogous manner.

The surface structuring preferably comprises first structural elements on the first connecting surface at least in segments and second structural elements on the second connecting surface at least in segments, which second structural elements are different from the first structural elements. The wrist endoprosthesis can be prevented from slipping in a particularly effective manner by providing various structural elements. Structural elements can thus be provided for the first connecting surface which are particularly suitable for a firm connection to human cartilage tissue, while second structural elements are provided for the second connecting surface, which enable a particularly firm connection to human bone tissue.

In a particularly preferred embodiment, the first structural elements are structural elements protruding from the first connecting surface. In the finished wrist endoprosthesis, the first structural elements then protrude in the direction of the anatomical structure. The protruding structural elements reduce a direct contact surface between the first connecting surface and the human tissue. The contact is then no longer flat, but rather only in the region of the contact elements. This can prevent tissue irritation. Furthermore, a manufacturing accuracy for the wrist endoprosthesis can be reduced in comparison to a flat contact. This is particularly desirable since only limited accuracies can often be achieved when acquiring data material. In this way, individual contact elements can be reworked more easily as part of an implantation than a large-area direct contact surface, and handling is made easier. The protruding structural elements nevertheless increase the total surface of the first connecting surface. A strength of a connection produced by a potting agent between the wrist endoprosthesis and the bony structure of the patient is thereby increased. Furthermore, distribution of the potting agent, particularly bone cement, is also promoted.

The first structural elements are preferably rhombuses, rectangles, triangles, circular structures, ellipses, polygons and/or ribs in cross section. The second structural elements are preferably pores in the second connecting surface. Pores allow bone tissue to grow in and thus ensure a particularly secure hold of the wrist joint prosthesis.

The wrist endoprosthesis preferably allows independent and/or externally controlled unfolding. For example, the wrist endoprosthesis can be implemented with joint segments for unfolding. Furthermore, the wrist endoprosthesis can preferably be produced from a reversibly deformable material. The size of wrist endoprostheses that allow unfolding can be reduced during implantation, so that such wrist endoprostheses can be implanted in a particularly patient-friendly manner. As soon as the wrist endoprosthesis is inserted into the wrist to be treated, said wrist endoprosthesis returns to the original shape thereof by unfolding. This can take place independently or automatically, that is, when deformation forces are withdrawn, or externally controlled with the aid of instruments provided for this purpose.

It should be understood that the wrist endoprosthesis according to the first consideration of the invention can also comprise a surface structuring on an anatomy-facing face, particularly on an anatomy-facing face of the carpal component. This can preferably be provided partially or completely on an inner surface of the carpal cavity. Likewise, the wrist endoprosthesis according to the first consideration of the invention can also preferably allow an independent or automatic and/or externally controlled unfolding.

In a further preferred embodiment, if for the case where the step of acquiring data material, particularly x-ray images, ultrasound images, magnetic resonance tomography data and/or computed tomography data, of a wrist of a patient to be treated is not possible, at least one of the following steps is carried out: acquiring data material, particularly x-ray images and/or computer tomography data, of the second wrist of a patient; and/or providing data from a statistical comparison group. It is not possible to acquire data material if the data material acquired is not suitable for further use in the method. However, it may be technically possible to acquire data that are not suitable for further use in the preferred method for producing a wrist endoprosthesis. Due to various clinical pictures or injury patterns, it may not make sense to acquire data material on the wrist to be treated, so that it is not considered possible in the context of the method. Determining parameters from data material of the wrist is useful if the data material comprises information and/or references to one or more features of the natural structure of the wrist of the patient in a particularly healthy state. A natural structure of the healthy wrist of the patient is approximated and/or simulated by individualizing the wrist endoprosthesis. Determining parameters from the data material does not make sense if the data material does not allow any conclusions to be drawn about the healthy wrist of the patient. For example, the joint structures can already be changed to such an extent due to rheumatoid arthritis that determining parameters from data material for said wrist does not make sense, since the data material does not contain any information and/or references to the healthy structure of the wrist. The wrist shows a healthy structure if a bony geometry is intact and said bony geometry allows a symptom-free use of the wrist. The data material of the statistical comparison group preferably contains data from comparison persons of the same age, same sex, having the same body size and/or having the same body type. The step of providing data material for a statistical comparison group is preferably carried out as an alternative or in addition if the acquisition of data material, particularly x-ray images and/or computed tomography data, of the second wrist of a patient is not possible or not sufficiently possible. Acquiring data material of the second wrist of a patient is not sufficiently possible, for example, if the data material only allows limited conclusions to be drawn about a natural structure of the wrist of the patient in a particularly healthy state due to various clinical pictures or injury patterns.

Furthermore, the method preferably comprises: providing or creating a 3D instrument model of one or more surgical instruments based on the individualized 3D model of the wrist endoprosthesis and/or the data material; and producing the surgical instrument based on the 3D instrument model. At least one of the surgical instruments is preferably an incision template which specifies an intraoperative cutting sequence. In the sense of a non-exhaustive list, incision templates for a joint surface of the radius, templates for orienting and guiding the drilling channel in the radius and/or rasps or files that are used to work on the bones of a patient can represent such preferred surgical instruments. The surgical instrument is preferably an instrument for supporting and/or implementing an unfolding of the wrist endoprosthesis.

In a further preferred form of the method, the data material contains at least data relating to one of the following parameters: ulnar variance, thickness of the cortex, plane shape of the inner cavity of the radius, radius of the proximal carpal row in a sagittal plane and/or frontal plane, radius of the distal carpal row in a sagittal plane and/or frontal plane, radius of one or more carpal bones in a sagittal plane and/or frontal plane, joint surface angle in a sagittal plane and/or frontal plane, course of the mechanical forearm axis, position points and/or orientation of anatomical axes of the carpal, position points and/or orientation of anatomical axes of individual carpal bones, width and/or length of one or more carpal bones, measured in a sagittal plane and/or a frontal plane, width of the proximal carpal row in a frontal plane and/or sagittal plane, width of the carpal in a frontal plane and/or sagittal plane, orientation of one or more carpal bones to a reference system and/or orientation of one or more carpal bones relative to one or more further carpal bones, diameter of the radius in a frontal plane and/or sagittal plane, anatomical position of a first transition from an epiphysis to a metaphysis of the radius, anatomical position of a second transition from the metaphysis to a diaphysis of the radius, width a proximal carpal row in the frontal plane and/or sagittal plane, height of a proximal carpal row, height of the carpal, frontal joint line, sagittal joint line and/or position of a longitudinal axis of the radius in extension through a third metacarpal bone, position of a first transition region from the epiphysis of the radius in the metaphysis of the radius. Ulnar variance represents a distal difference in length between the radius and ulna of a patient. An extension from a proximal end of the carpal or carpal bone to a distal end of the carpal or carpal bone is referred to as length. The width of the carpal or carpal bone is an extension perpendicular to the length. A joint line preferably bisects a distance between the proximal carpal row and the joint surface of the radius and/or the joint surface of the ulna.

Furthermore, the step of individualizing the 3D model preferably comprises at least one of the following steps: adapting an angle of a first joint surface to the derived joint surface angle in a sagittal plane; adapting an angle of a second joint surface to the derived joint surface angle in a sagittal plane; adapting an angle of a first joint surface to the derived joint surface angle in a frontal plane; adapting an angle of a second joint surface to the derived joint surface angle in a frontal plane; adapting a radius of a convex elevation of a carpal component to the derived radius of the proximal carpal row in a sagittal plane and/or frontal plane; adapting a size of a distal part of a radius component such that protruding of the distal part beyond a radius of a patient is avoided or adapted; adapting a diameter and/or a shape of a shaft of a radius component as a function of the derived radius diameter of a patient, preferably taking into account the thickness of the cortex and/or a jamming allowance; adapting the maximum clear width of the carpal cavity of the carpal component to the width of the proximal carpal row in the sagittal plane and/or the width of the carpal in the sagittal plane; adapting the corresponding clear width of the carpal cavity of the carpal component to the width of the proximal carpal row in the sagittal plane and/or the width of the carpal in the sagittal plane; adapting a length of the carpal component, measured in the distal-proximal direction, to a length of the proximal carpal row; adapting a maximum depth of the carpal cavity to the length of the proximal carpal row; aligning the carpal component and the radius component substantially perpendicular to the position of the longitudinal axis of the radius in extension through the third metacarpal bone; adapting a surface of the shaft of the radius component such that the surface of the shaft is porous and/or comprises unevenness in a region from the connecting segment of the radius component to the first transition region; adapting the first and/or second joint surface of the wrist endoprosthesis to a joint line.

In a preferred refinement of the method, a first material used to produce the wrist endoprosthesis is an isoelastic material, preferably a plastic, particularly preferably PEEK. Furthermore, the first material can preferably be supplemented by a second material, preferably titanium. A surface formed by the first material is preferably completely or partially coated with the second material.

In a fifth consideration, the invention achieves the object named above with a method for producing a wrist endoprosthesis, particularly a wrist prosthesis according to one of the embodiments described above according to the first consideration of the invention, wherein the method comprises the steps: providing or producing a non-individualized 3D model of a wrist endoprosthesis; acquiring data material, particularly x-ray images, magnetic resonance tomography data, ultrasound images and/or computed tomography data, of a wrist of a patient to be treated; creating a patient-specific 3D model of at least one anatomy segment of the wrist to be treated using the data material; deriving a negative shape of the anatomy segment of the wrist to be treated; adapting an anatomy-facing face of the non-individualized 3D model to the derived negative shape of the anatomy segment of the wrist to be treated for obtaining an individualized 3D model; and producing the wrist endoprosthesis by means of an additive manufacturing process based on the individualized 3D model. The face facing the anatomy preferably reproduces the negative shape substantially identically. However, tolerance allowances can particularly preferably also be provided. The face facing the anatomy can thus preferably be an envelope surface of the negative shape that is spaced apart by a tolerance distance.

Furthermore, in a sixth consideration, the invention achieves the object named above by means of a computer program product comprising code means which, when executed on a computer, are implemented to execute one of the steps of the previously defined method according to the fourth and/or fifth consideration of the invention. It should be understood that the wrist endoprosthesis according to one of the first, second and third considerations, the method according to the fourth and fifth consideration of the invention and the computer program product according to the sixth consideration of the invention, have the same and similar sub-considerations as set out particularly in the dependent claims, so that reference is also made in full to the above description for the further preferred embodiments of the computer program product.

The invention is explained in more detail below using an exemplary embodiment with reference to the accompanying figures. Said figures are not necessarily intended to depict the embodiment to scale; rather, the drawing is shown in schematic and/or slightly distorted form for explanatory purposes. With respect to supplements to the teachings directly discernible from the drawing, reference is made to the applicable prior art. It must be taken into account that various modifications and changes relating to the shape and detail of an embodiment can be made without deviating from the general idea of the invention. The features of the invention disclosed in the description, in the drawing, and in the claims can be essential to the refinement of the invention individually and in any arbitrary combination. In addition, all combinations of at least two of the features disclosed in the description, the drawing and/or the claims fall within the scope of the invention. The general idea of the invention is not limited to the precise form or the detail of the preferred embodiment shown and described below or limited to a subject-matter that would be limited in comparison with the subject-matter claimed in the claims. In the case of the specified measurement ranges, values lying within the stated limits should also be disclosed as limit values and be able to be used and claimed as required. For simplicity, identical reference numerals are used below for identical or similar parts or parts having identical or similar functions.

Further advantages, features and details of the invention emerge from the following description of the preferred embodiment and with reference to the drawings; these show:

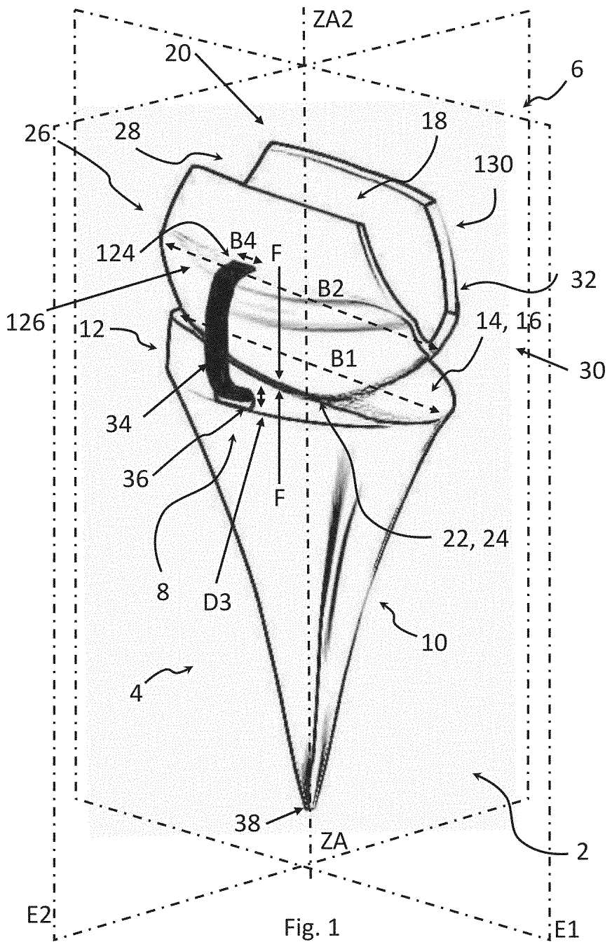

FIG. 1 an isometric view of the wrist endoprosthesis that has the luxation protection attached;

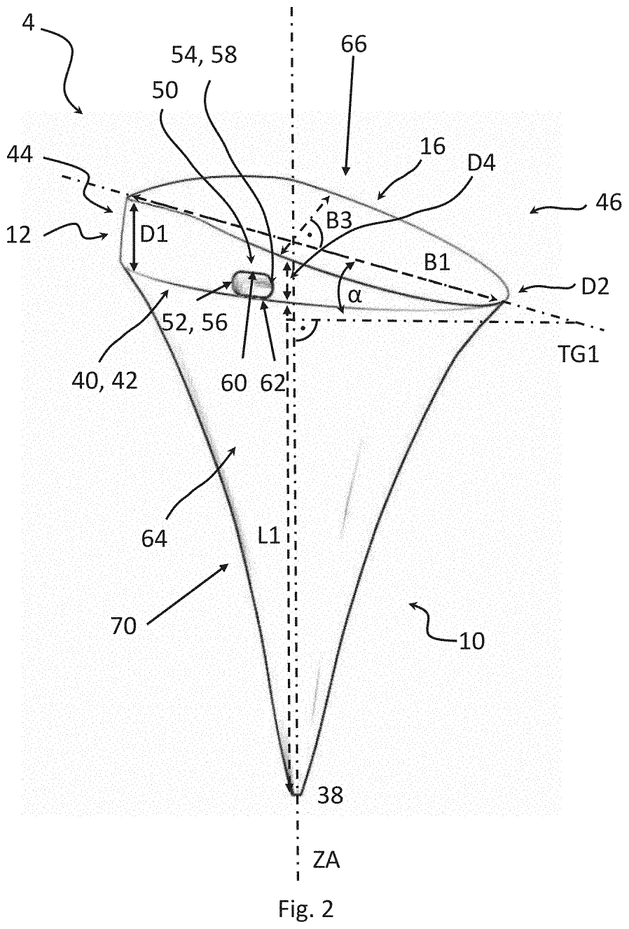

FIG. 2 an isometric view of the radius component;

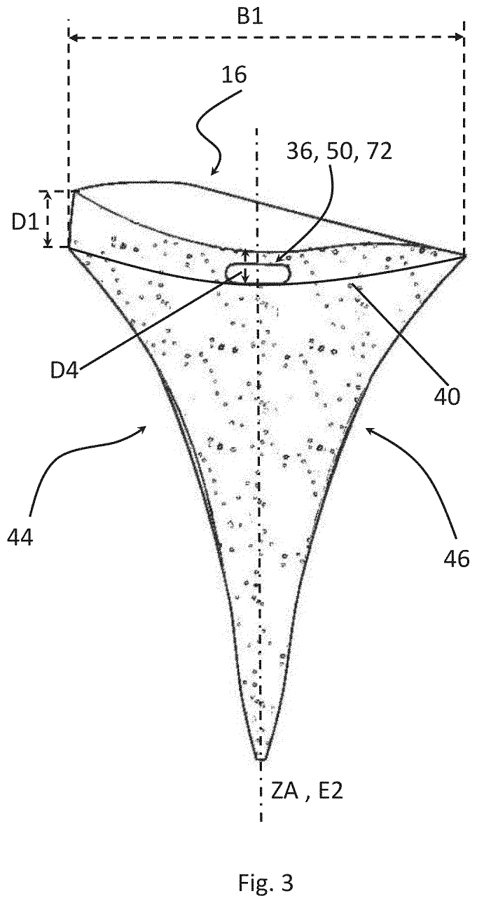

FIG. 3 a section of the radius component along the frontal plane;

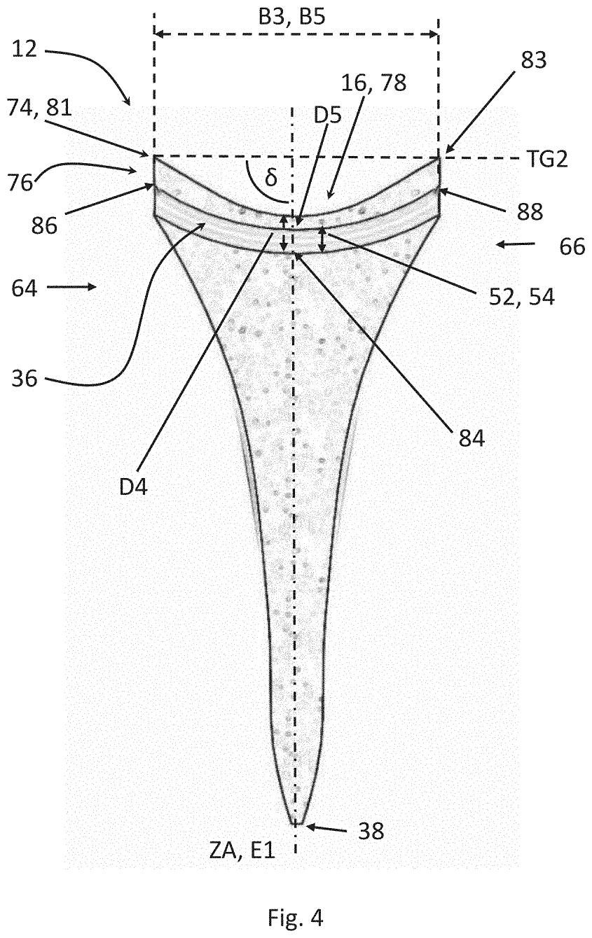

FIG. 4 a section of the radius component along the sagittal plane;

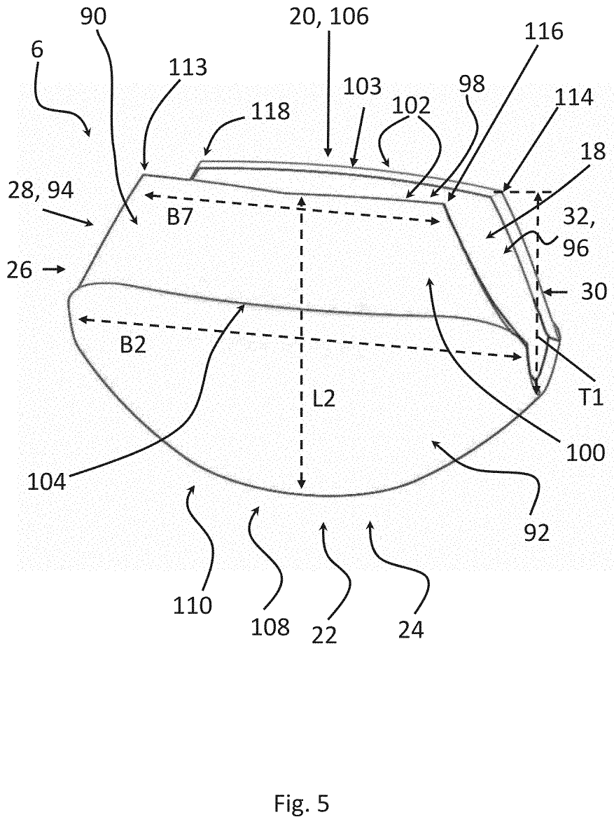

FIG. 5 an isometric view of the carpal component;

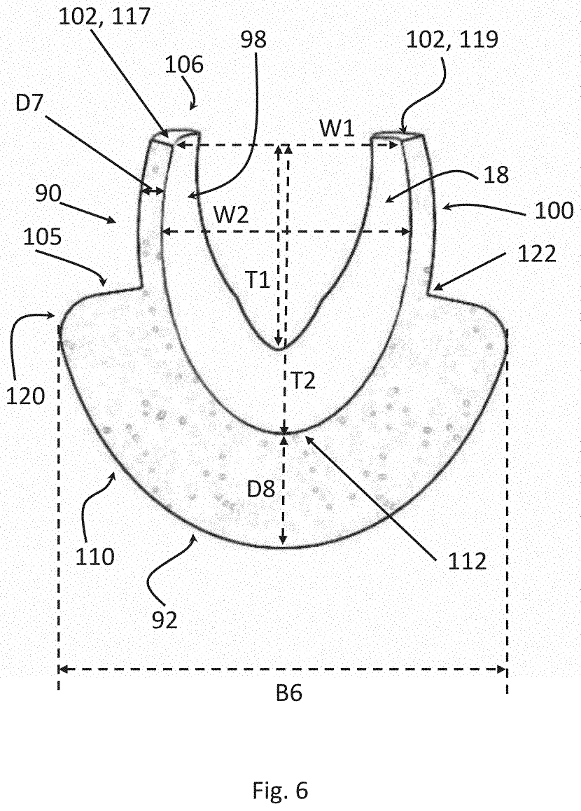

FIG. 6 a section of the carpal component in the sagittal plane;

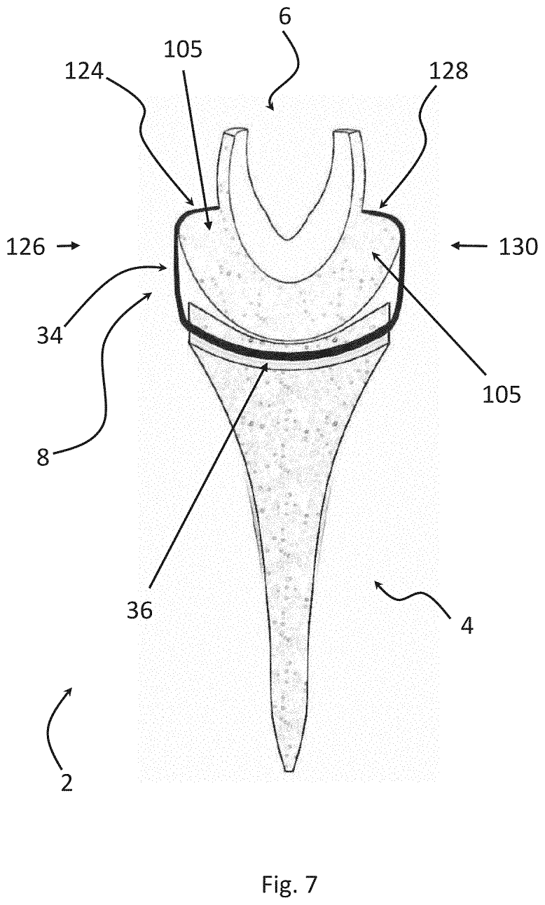

FIG. 7 a section of the wrist endoprosthesis along the sagittal plane;

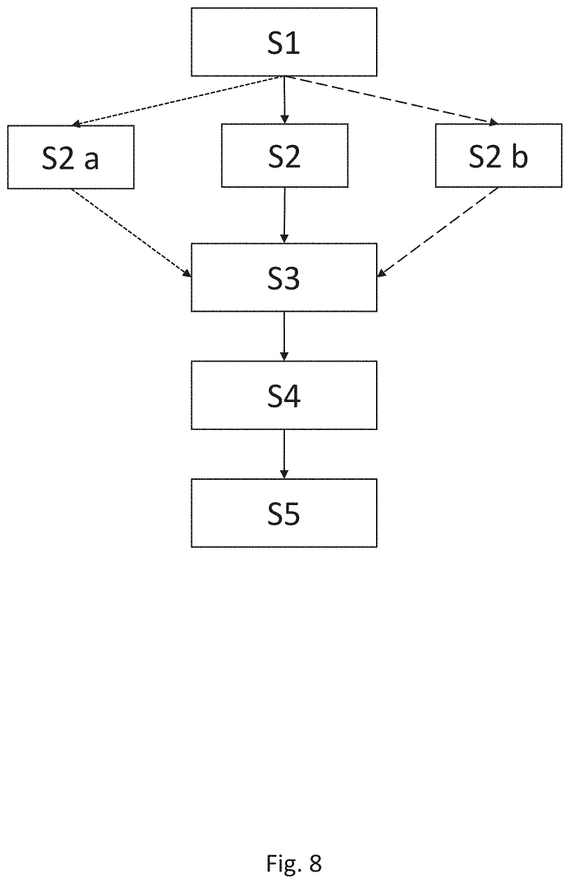

FIG. 8 a flow diagram illustrating the method for producing a wrist endoprosthesis;

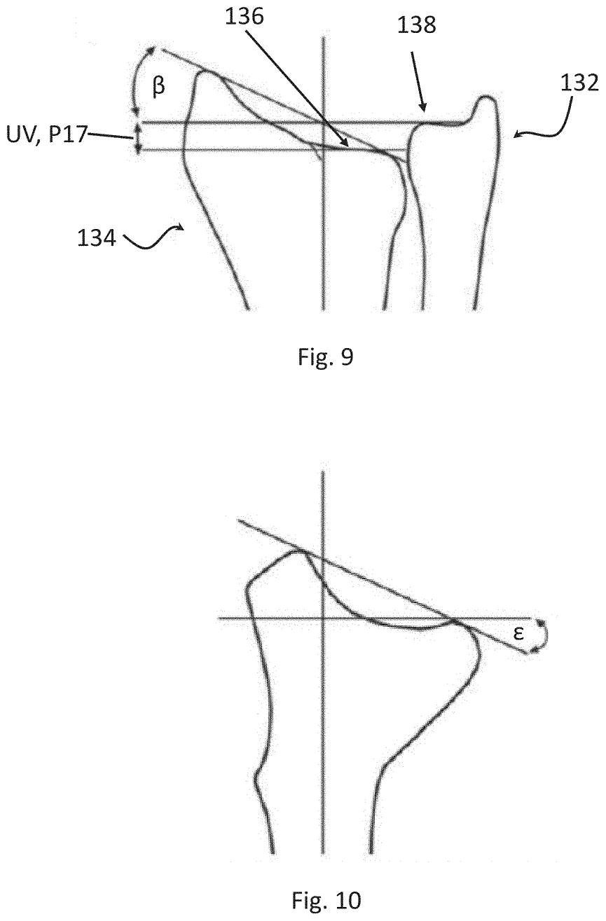

FIG. 9 a dorsal view of the human bones of the right forearm;

FIG. 10 a view of the human radial bone in the sagittal plane;

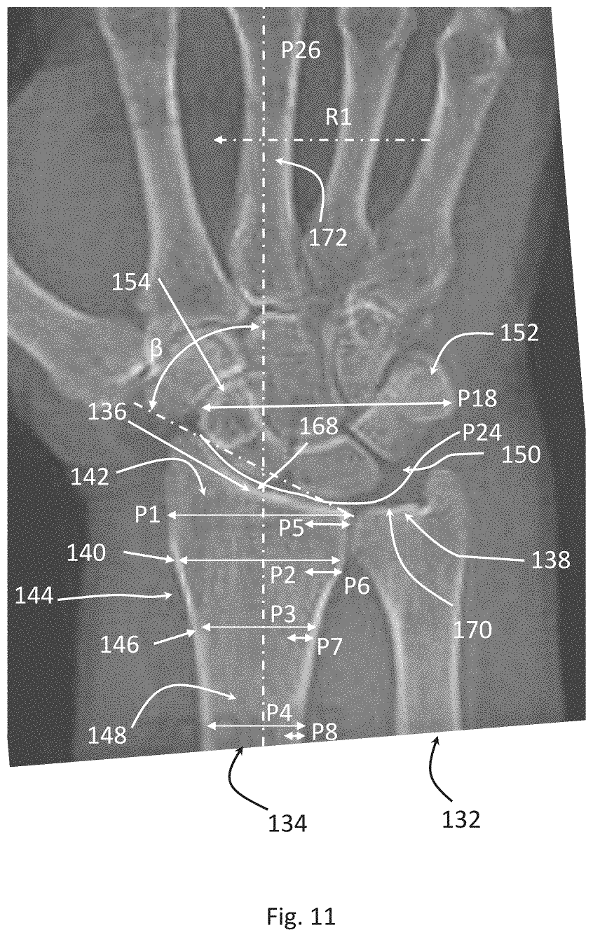

FIG. 11 an anterior-posterior view of the bone structure of a human wrist;

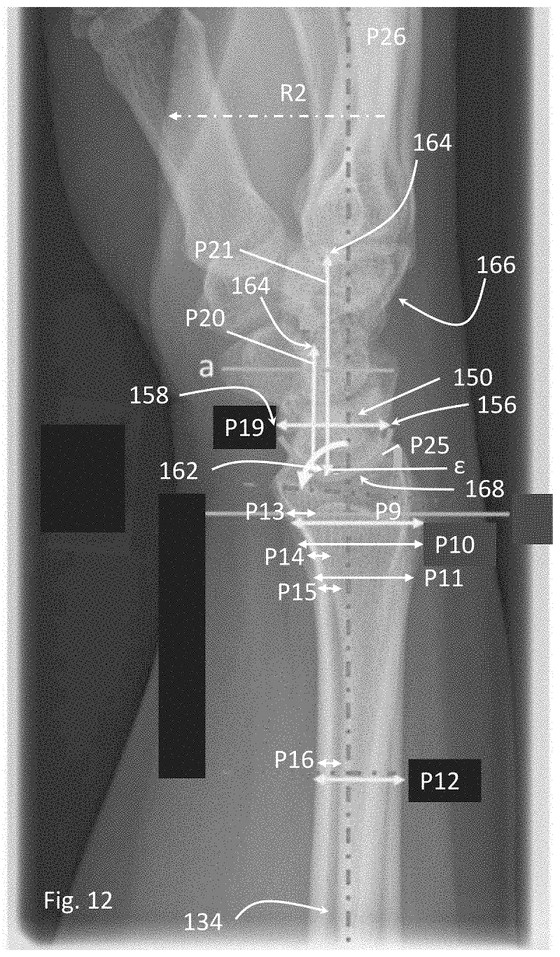

FIG. 12 a view of the bone structure of a human wrist in a sagittal plane; and

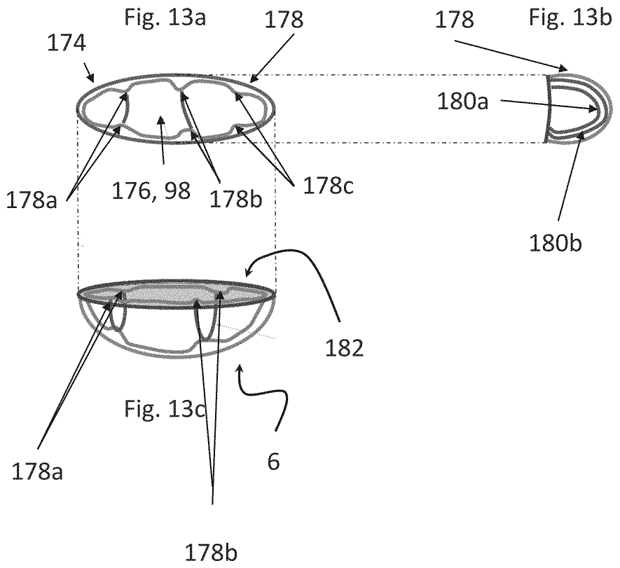

FIG. 13a-13c schematic views illustrating an individualized 3D model of a wrist endoprosthesis.

According to the present embodiment example (FIG. 1), a wrist endoprosthesis 2 comprises a radius component 4, a carpal component 6 and luxation protection 8 as main elements. The radius component 4 is formed here by a shaft 10 and a head 12. A first joint surface 16 is disposed on a distal head face 14. The carpal component 6 is formed trough-shaped and preferably comprises a carpal cavity 18. In the present embodiment, the trough shape of the carpal component 6 is formed by the carpal cavity 18. The carpal cavity 18 is open to a distal carpal face 20. A second joint surface 24 is disposed on a proximal carpal face 22 which is opposite the distal carpal face 20. In the present embodiment example, there is contact between the first joint surface 16 and the second joint surface 24. It should be understood that wrist endoprostheses 2 that have a distance between the first joint surface 16 and the second joint surface 24 are also preferred. The carpal component 6 has a first lateral opening 28 on a radius face 26. The carpal component 6 comprises a second lateral opening 32 on an ulnar face 30 opposite the radius face 26.

According to the present embodiment example, the wrist endoprosthesis 2 comprises luxation protection 8 which is formed by a band 34 which connects the radius component 4 and the carpal component 6. The band 34 runs through a tunnel 36 formed in the radius component. The carpal component 6 is preferably disposed in the direction of the central axis ZA on the distal head face 14. In the embodiment described, the central axis ZA runs from a proximal shaft end 38 to the distal head face 14. A second central axis ZA2 of the carpal component 6 is congruent to the central axis ZA in the present embodiment example. It should be understood that the central axis ZA and the second central axis ZA2 are at a distance and/or can include an angle with one another. A first maximum width B1 of the radius component, measured parallel to the frontal plane E1, is preferably greater than or equal to a corresponding second maximum width B2 of the carpal component 6. The second maximum width B2 preferably has a range from 70% to 100%, preferably 85% to 100%, particularly preferably 90% to 95% of the first maximum width B1. In the exemplary embodiment shown, the luxation protection 8 applies a restoring force F, which causes contact between the first joint surface 16 and the second joint surface 24. However, it should be understood that luxation protection 8 is also preferred which does not apply any restoring force in a rest position. For example, the band 34 of the luxation protection 8 could be implemented as a rigid band that counteracts longitudinal expansion.

Referring now to FIG. 2, the radius component 4 of the present embodiment will be explained. The shaft 10 of the radius component 4 is implemented to be implanted in a radius of a patient. The head 12 protrudes from a distal end of the radius. In the preferred embodiment, the head 12 extends in the direction of the central axis ZA from the shaft 10 to the first joint surface 16. The head 12 is connected to the shaft 10 at a connecting segment 40. The connecting segment 40 is preferably implemented as an edge 42. It should be understood that rounded and/or continuous connecting segments 40 are also preferred. In the embodiment shown, the connecting segment 40 is substantially circular in a plane perpendicular to the central axis ZA. Substantially oval or egg-shaped connecting segments 40 are also preferred.

A thickness D1 on a radius face 44 of the radius component 4, measured in the direction of the central axis, is greater than a thickness D2 on an ulnar face 46. In the embodiment shown, the first joint surface 16 merges directly into the shaft 10 on the ulnar face 46 and the thickness D2 disappears. However, thicknesses D2 of the head 12 in the region of the ulnar face 46 which are greater than zero are also preferred. In the frontal plane E1 (see FIG. 1), a tangent TG1 to the first joint surface 16 includes an angle α with a plane perpendicular to the central axis ZA. The angle α has a range from 0° to 60°, preferably 0° to 45°, particularly preferably 15° to 20°. Negative angles α are also preferred, so that the thickness D2 is greater than the thickness D1. The first joint surface 16 can preferably also comprise planar segments. In the embodiment shown, the first joint surface 16 is implemented substantially oval, so that the width B1 is greater than a third width B3 of the joint surface 16 that is perpendicular to the first width B1.

The tunnel 36 comprises a first access 50 at the head 12 of the radius component 4. According to the present embodiment example, the first access 50 is disposed on the dorsal face 64 of the radius component 4. A second access 51 is preferably disposed on the palmar face 66 of the radius component 4. The second access 51 is preferably mirrors symmetrical to the first access 50 in the frontal plane. In the embodiment shown, the first access 50 has a cross section in the form of an elongated hole. The narrow faces 52, 54 of the first access 50 are closed by semicircles 56, 58. The long faces 60, 62 of the first access 50 are parallel and larger than the narrow faces 52, 54. Here, a width B4 of the band 34 is less than a length of the long faces 60, 62 and/or a thickness D3, measured perpendicular to the width B4, of the band 34 is less than a length of the narrow faces 52, 54. Round, oval and/or rectangular cross sections of the first access 50 and/or second access 51 are also preferred. The accesses 50, 51 can be rounded in order to minimize the loads on the band 34. The first access 50 and the second access 51 are preferably disposed completely on the head 12 of the radius component 4. In the embodiment shown, the first access 50 directly adjoins the connecting segment 40 in the direction of the distal head face 14. It should be understood that embodiments are also preferred in which the first access 50 is implemented at a distance from the connecting segment 40 in the direction of the central axis ZA. The first access 50 particularly preferably has a smaller distance from the connecting segment 40 in the direction of the central axis ZA than from the first joint surface 16.

According to the present embodiment example, the shaft 10 tapers continuously in the direction of the central axis ZA towards the proximal shaft end 38. Embodiments that have a shaft 10 alternately tapering and widening in the direction of the proximal shaft end are also preferred. In the embodiment shown, the proximal shaft end 38 is shown flattened. Pointed and/or rounded proximal shaft ends 38 are also preferred. According to the present embodiment example, a lateral surface 70 of the shaft 10 is implemented continuously. The shaft 10 can preferably be implemented to be rotationally symmetrical to the central axis ZA. The shaft 10 preferably has a length L1, measured along the central axis ZA between the connecting segment 40 and the proximal shaft end 38, in a range from 10 mm to 150 mm, particularly preferably 50 mm to 100 mm. A thickness D4 of the head 12 adjoining the length L1 along the central axis ZA has a range from 0 mm to 100 mm, preferably 10 mm to 40 mm, particularly preferably 20 mm to 30 mm.

FIG. 3 shows a section through the radius component 4 along the frontal plane E1. In the embodiment described, the first joint surface 16 is implemented alternately concave and convex in the frontal plane E1. Purely concave or purely convex courses of the first joint surface 16 are also preferred. The tunnel 36 preferably has a constant cross section 72 which corresponds to the cross section of the first access 50. The cross section 72 is preferably implemented mirror-symmetrically to the central axis ZA. The maximum width B1, measured perpendicular to the central axis ZA, is disposed in the transition region 40 in the embodiment example. It should be understood that embodiments that have maximum widths B1 along the central axis ZA in the direction of the first joint surface 16 from the transition region 40 are also preferred.

FIG. 4 shows a section through the radius component 4 along a sagittal plane E2. In the embodiment shown, an upper transition region 74 between the first joint surface 16 and a face surface 76 of the head 12 is implemented as an edge. Furthermore, completely or partially rounded transition regions 74 are also preferred. According to the present embodiment example, the first joint surface 16 is implemented in the sagittal plane E2 as a concave recess 78 which runs along the central axis ZA in the direction of the proximal shaft end 38. Likewise, first joint surfaces 16 which are implemented as convex elevations are also preferred.

In the embodiment described, the tunnel 36 runs in the region of the head 12 from the dorsal face 64 of the radius component 4 to the palmar face 66 and is curved in the direction of the proximal shaft end 38. A proximal point 84 of the tunnel 36, measured in the direction of the central axis ZA, is preferably disposed on the central axis ZA. The proximal point 84 is that point of the tunnel 36 which, measured in the direction of the central axis ZA, has the smallest distance from the proximal shaft end 38. Distal transition regions 86, 88 of the tunnel 36 to the dorsal face 64 and palmar face 66 are preferably rounded. Fully rounded first and second accesses 50, 51 are also preferred. The material of the head 12 between the joint surface 16 and the tunnel 36 has a minimum thickness D5 in a range of 20% to 200%, preferably 30% to 150%, particularly preferably 40% to 110%, of the length of the narrow face 52,54 of the tunnel 8. Furthermore, the minimum thickness D5 preferably has a range from 10% to 60%, preferably 50% to 60% of the thickness D4. Furthermore, the thickness D5 is preferably greater near the transition regions 86, 88 than in the region of the central axis ZA.

In the embodiment described, the radius component 4 is implemented mirror-symmetrically to the frontal plane E1. It should be understood that non-symmetrical or component-wise symmetrical embodiments of the radius component 4 are also preferred. For example, only the tunnel 36 and/or the transition region 40 could be implemented symmetrically to the frontal plane E1. A tangent TG2 is implemented tangentially to delimitation points 80, 82 of the first joint surface 16 in the sagittal plane E2. According to the present embodiment example, an angle δ between the tangent TG2 and a plane perpendicular to the central axis ZA is 90°. It should be understood that angles δ are preferred which have a range from 30° to 150°, particularly preferably 80° to 120°.

According to the present embodiment example, the first joint surface 16 is implemented concave in the sagittal plane E2. Concave first joint surfaces 16 in the frontal plane E1 are also preferred. The face surface 76 of the head 12 is formed on the dorsal face 64 of the radius component 4 and the palmar face 66 substantially parallel to the central axis ZA, so that the width of the head 12 in the sagittal plane E2 is constant and corresponds to the width B3 of the first joint surface 16. Embodiments are also preferred in which the head 12 widens and/or tapers in the direction of the first joint surface 16 so that a maximum width B5 differs from the width of the joint surface B3. The width B3 has a range from 10% to 150%, preferably 50% to 100%, particularly preferably 70% to 90%, of the width B1 in the frontal plane E1. In the embodiment shown, an angle δ, measured between the central axis ZA and a tangent TG1 at a first end point 81 on the dorsal face 64 and a second end point 83 on the palmar face 66, is 90°; other angles in a range of 0° to 180° are also preferred.

Referring now to FIGS. 5 and 6, the carpal component 6 will be described in more detail. FIG. 6 shows a section through the carpal component 6 in the sagittal plane E2. The carpal component 6 comprises a distal segment 90 and a proximal segment 92. The carpal cavity 18 is open to a distal carpal face 20. A second joint surface 24 is implemented on a proximal carpal face 22 opposite the distal carpal face 20. According to the present embodiment example, a maximum width B2 of the carpal component 6 parallel to the frontal plane E1 is greater than a maximum width B6 of the carpal component 6 perpendicular thereto in the sagittal plane E2 (see FIG. 6). The carpal component 6 is implemented elongated parallel to the frontal plane E1. In the embodiment shown, the first lateral opening 28 disposed on the radius face 26 is implemented as a first slot 94. In an analogous manner, the second lateral opening 32 disposed on the ulnar face 30 is implemented as a second slot 96. The slots 94, 96 are open in the direction of the distal carpal face 20.

In the embodiment shown, the first slot 94 and the second slot 96 are implemented mirror-symmetrically to the sagittal plane E2. A depth T1 of the slots 94, 96, measured starting from the distal carpal face 20 in the direction of the proximal carpal face 22, extends into the proximal segment 92. Likewise, the slots 94, 96 can only be disposed in the distal segment 90 of the carpal component 6. The depth T1, measured between the distal carpal face 20 and the proximal carpal face 22, has a range from 20% to 80%, preferably 30% to 70%, particularly preferably 40% to 60%, of the length L2 of the carpal component 6. It should be understood that asymmetrically implemented slots 94,96 are also preferred. The first slot 94 can preferably have a third depth T3, which is different from the depth T1. It is further preferred that the carpal cavity 18 comprises a bulge 112 in the direction of the proximal carpal face 22. A depth T2 of the bulge, measured from the distal carpal face 20 in the direction of the proximal carpal face 22, is preferably greater than the depth T1 and/or the depth T3. In the embodiment shown, the transition regions 102 between an inner surface 98 of the carpal cavity and an outer surface 100 of the carpal component 6 are implemented with edges 103. Rounded transition regions 102 are also preferred.

In the embodiment shown, the transition 104 between the distal segment 90 and the proximal segment 92 is implemented as a shoulder 105. The distal segment 90 of the carpal component 6 is parallel to the frontal plane E1, tapering in the direction of the distal carpal face 20, so that said distal segment has a trapezoidal shape in the present embodiment example. Outer corners 113,114,116,118 are implemented pointed here. Rounded corners 113,114,116,118 are also preferred. A width B7 parallel to the frontal plane at the distal end 106 of the carpal component 6 is less than a maximum width B2. The proximal end 108 of the carpal component 6 is disposed on the proximal face 22. The proximal end 108 is preferably implemented as a thickening 110. The second joint surface 24 is disposed on the thickening 110. The thickening 110 is implemented here as a convex elevation. The thickening can also be implemented as a concave recess, so that the second joint surface 24 is also concave in the frontal plane E1. According to the present embodiment example, the thickening 110 extends over the entire proximal segment 92 of the carpal component 6. In addition, embodiments that have a thickening 110 that extends only partially over the proximal segment 92 are also preferred. A thickness D7 of the carpal component 6, measured between the inner surface 98 and the outer surface 100 in the region of the distal end 106, is here less than a corresponding thickness D8 in the region of the proximal end 108. A ratio of the wall thicknesses D8:D7 preferably has a range from 8:1 to 1:1, more preferably 6:1 to 1:1, particularly preferably 6:1 to 3:1.

The carpal cavity 18 has a substantially U-shaped cross section. According to the present embodiment example, a clear width W1 of the carpal cavity 18 at the distal end 106 is smaller than a maximum clear width W2 in the interior of the carpal cavity 18. The carpal cavity 18 preferably tapers in the direction of the distal carpal face 20. Carpal cavities 18 which widen in the direction of the distal carpal face 20 are also preferred. In the embodiment described, the transition regions 102 at the distal end of the carpal component 6 are implemented as planar surfaces 117, 119. A rounded transition between inner surface 98 and outer surface 100 is also preferred.

In the exemplary embodiment, the second joint surface 24 is convex in the sagittal plane E2. However, concave or concave-convex second joint surfaces are also preferred. Here, a first transition 120 between shoulder 105 and the proximal segment 92 is rounded, while a second transition 122 between the shoulder 105 and the distal segment 90 is angled, particularly right-angled. Two rounded or angular transitions 120, 122 and a continuous transition without a shoulder 105 between the distal segment 90 and the proximal segment 92 are also preferred. If the first transition 120 and/or the second transition 122 are rounded, impairment of the surrounding soft tissue of a patient can be reduced or avoided after the implantation. The proximal end 108 of the carpal component 6 preferably comprises a continuous and/or rounded outer surface for this purpose.

Referring now to FIG. 7, the luxation protection 8 will be further described. The band 34 of the luxation protection 8 comprises a first end 124 which is attached to the dorsal carpal face 126. Furthermore, a second end 128 of the band 34 is attached to a palmar carpal face 130. The dorsal carpal face 126 is preferably substantially perpendicular to the distal carpal face 20 and to the radius face 26. In the present embodiment example, the first end 124 and the second end 128 are attached to the shoulder 105. It is also preferred that the first end 124 and/or the second end 128 are attached to the distal end 106 of the carpal part 6, to the distal segment 90, to the proximal segment 92, to the proximal end 108 and/or to the inner surface 98 of the carpal cavity 18. The attaching can preferably take place in a positive, frictional and/or material manner. The band 34 extends through the tunnel 36 between the first end 124 and the second end 128. The carpal component 6 and the radius component 4 of the wrist endoprosthesis 2 are thereby connected. The band 34 is only connected to the radius component 4 in a positive manner here by a loop formed between the first end 124 and the second end 128. The band 34 can thereby preferably slide relative to the tunnel 36. Rotational and tilting movements of the carpal component 6 relative to the radius component 4 are possible. The frictional forces between band 34 and tunnel 36 and between the first joint surface 16 and the second joint surface 24 must be overcome for this purpose. Synovial fluid can act as a lubricant. Furthermore, embodiments that have a fixed connection of the band 34 to the radius component 4 are preferred.

The band is preferably formed from a fibre material and/or a plastic. The band 34 is preferably pretensioned such that said band applies a pretensioning force F which brings about a contact between the first joint surface 16 and the second joint surface 24. A lifting of the carpal component 6 from the radius component 4 can be avoided by the pretensioning force. The pretensioning force has a range from 0 N to 1000 N, preferably 5 N to 200 N, particularly preferably 100 N to 150 N.

Embodiments that comprise a rigid band 34 are also preferred. A rigid band 34 has a high resistance to longitudinal expansion and thus counteracts the longitudinal expansion thereof. The pretensioning force can be reduced or omitted, as a result of which frictional forces occurring between the first joint surface 16 and the second joint surface 24 are reduced. A slight gap between the first joint surface 16 and the second joint surface 24 is possible.

Furthermore, embodiments of the invention that have a second band running through a second tunnel are preferred (not shown). The second tunnel preferably runs perpendicular to the first tunnel 36, wherein a first end of the second band is attached to a radius face 26 of the carpal component 6 and a second end of the second band is attached to an ulnar face 28 of the carpal component 6. The carpal component 6 can be prevented from slipping off the first joint surface 16 by designing the luxation protection 8 that has two bands. Furthermore, embodiments are preferred in which the head 12 does not comprise a tunnel (not shown). In such embodiments, a first end 124 of the band 34 can be connected to the carpal part 6 and a second end 128 of the band 34 can be connected to the radius part 36.

The further embodiments can have the same or similar features as the embodiment according to the example described, which is why reference is made in full to the above description.

The flow chart shown in FIG. 8 illustrates a method for producing a wrist endoprosthesis 2. In a first step S1, a non-individualized 3D model of a wrist endoprosthesis 2 is provided or produced. The non-individualized 3D model is preferably produced using computer-aided image processing programs and/or 3D CAD programs. Providing the non-individualized 3-D model can preferably have the following steps: selecting a suitable non-individualized 3D model from a database and loading the selected non-individualized 3D model into an image processing program and/or 3D CAD program.

In a second step S2, data material, particularly x-ray images, ultrasound images, magnetic resonance tomography data, and/or computed tomography data, of a wrist of a patient to be treated is acquired. The data material is preferably acquired at least in a first view parallel to the frontal plane E1 and in a second view parallel to the sagittal plane E2. Further views at an angle to the first and second views are preferably acquired. Furthermore, a 3D model of the wrist to be treated is preferably created from the magnetic resonance tomography data and/or computed tomography data. If information on the wrist to be treated is no longer available due to injuries or previous illnesses, step S2a is preferably carried out: acquiring data material, particularly x-ray images, ultrasound images, magnetic resonance tomography data and/or computer tomography data, of the second wrist of a patient. Data material which corresponds to the data material from step S2 is preferably acquired, which is why reference is made in full to the above description. If information from both wrists of the treating patient is no longer available, step S2b is preferably carried out: providing data from a statistical comparison group. The data material of the statistical comparison group can contain information equivalent to that of the data material acquired in steps S2 or S2a.

The comparison group can have people of the same sex, same age, same body size, same arm length, same hand size and/or the same anthropometric data. It should be understood that steps S2, S2a and S2b can also be executed in combination. For example, a first joint surface of the radius can be determined from x-ray images of the wrist to be treated and an angle of a second joint surface can be determined from data material from a statistical comparison group. Alternative or supplementary data material can preferably be provided which is based on individual empirical values, simulation data, literature data, statistical models, anthropometric comparison data, anthropometric ratio calculations, geometric ratios, previous examinations of the patient and/or mathematically determined optima.

Following steps S2, S2a and/or S2b, step S3 is carried out in the method: determining one or more parameters from the data material for approximating an approximated joint structure of the patient from the data material. The parameters can be determined from the data material manually, partially or fully automatically, preferably based on image material or 3D models. The parameter determined from the data material can be at least one of the parameters from the following group of parameters: ulnar variance UV, thickness of the cortex, plane shape of the inner cavity of the radius, radius of the proximal carpal row in a sagittal plane and/or frontal plane, joint surface angle in a sagittal plane and/or frontal plane, course of the mechanical forearm axis, curve course of the proximal and/or distal carpal row in a sagittal plane and/or frontal plane. The ulnar variance UV describes a length difference between the segment of the distal end 136 of the radius 134 pointing towards the ulna 132 and the segment of the distal end 138 of the ulna 132 of the arm to be treated pointing towards the radius 134. It is also possible to derive the course of the proximal and/or distal carpal row as an exact curve or to approximate said curve course as a radius or a curve course composed of a plurality of radii. The plane shape of the joint surfaces of the radial bone 134, the ulna 132 and/or the proximal carpal row can be derived exactly or approximately from the data material. The exact plane shape is preferably approximated by circular and/or elliptical fittings. Furthermore, the joint surfaces can be approximated by multi-axis convex, multi-axis concave or multi-axis convex-concave planes. Furthermore, an angle δ is preferably determined between a straight-line tangent to the distal end of the radius in the frontal plane and a straight line of the radius that is perpendicular to the longitudinal axis and lies in the frontal plane. In an analogous manner, an angle c is preferably also determined between a straight-line tangent to the distal end of the radius in the sagittal plane and a straight line that is perpendicular to the longitudinal axis of the radius and lies in the sagittal plane. The parameters of the data material such as plane shapes and surface shapes are preferably approximated. The approximation preferably includes at least the step: rounding the determined sizes to sizes that can be processed in the method. It is also preferred to execute step S1 following one of steps S2, S2a, S2b or S3.

In a fourth step S4, the non-individualized 3D model is individualized based on the derived parameters for obtaining an individualized 3D model.

Geometric properties of the non-individualized 3D model are preferably adapted to the approximated parameters. The angle α of the first joint surface is preferably adjusted or approximated to the determined or approximated angle R. Furthermore, the angle δ is preferably adjusted or approximated to the determined or approximated angle c. Furthermore, the length L1 and the tapering of the shaft 10 are preferably adapted taking into account at least one of the determined or approximated sizes, thickness of the cortex, diameter of the radius of a patient, diameter ratio of the radius. Furthermore, the first joint surface is preferably individualized such that said joint surface corresponds to the exact or approximated distal surface of the radius of the patient to be treated. In a further preferred embodiment of the method, the second joint surface is individualized such that said joint surface corresponds to the approximated and/or exact curve shape of the proximal carpal row of the patient in the sagittal plane and/or frontal plane.

Subsequent to step S4, step S5 is executed: producing the wrist endoprosthesis 2 by means of an additive manufacturing process based on the individualized 3D model. The additive manufacturing process is preferably one of the following processes: fused deposition modelling, selective laser sintering, selective laser melting, electron beam melting, laser deposition welding, multi-jet modelling, stereolithography, laminated object modelling. Step S5 preferably comprises at least one of the steps: loading the individualized 3D model, generating the layer information of the individual layers based on the individualized 3D model, generating the layer information of the individual layers to be manufactured taking into account support structures, preparing the manufacturing machine and/or the material, constructing the wrist endoprosthesis 2 layer-by-layer, removing the support structures, reworking the upper and/or active surfaces, checking for damage and/or compliance with the individualized 3D model, completely or partially coating the wrist endoprosthesis with a coating material, preferably titanium. A part of the shaft 10 which is disposed in the epiphysis 142 and/or the metaphysis 144 of the radius 134 as part of the implantation of the wrist endoprosthesis 2 is particularly preferably coated. The wrist endoprosthesis 2 is preferably made from a metal, fibre composite material and/or plastic, particularly preferably polyetheretherketone, cobalt-chromium alloys and/or titanium.

Referring now to FIG. 11 and FIG. 12, steps S3 and S4 of a preferred refinement of the method for producing a wrist endoprosthesis 2 are described. The anterior-posterior view of the bone structure of a human wrist shown in FIG. 11 and the view of the bone structure of a human wrist shown in FIG. 12 in a sagittal plane were previously acquired in step S2.

First, parameters P1 to P16 are determined, which parameters preferably form a basis for an individualization of the shaft 10 of the radius component 4 carried out in step S4. The parameters P1 to P4 represent frontal radius diameters of the radius 134, which are parallel to one another and are determined perpendicular to a central axis of the radius 134 in the frontal plane E1. The frontal radius diameter P1 is preferably determined at the height of the distal end 138 of the ulna 132. A second frontal radius diameter P2 is preferably determined at a first transition 140 between an epiphysis 142 and a metaphysis 144 of the radius 134 and/or a third frontal radius diameter P3 is determined at a second transition 146 between the metaphysis 144 and a diaphysis 148 of the radius 134. A position of the fourth frontal radius diameter P4 is determined individually for each patient, based on the bone structure of the patient. Further frontal radius diameters can preferably also be determined from the data material. Preferably, the thickness of the cortex of the radius 134, measured in the frontal plane E1, is determined as parameters P5 to P8 at the height of the distal end 138 of the ulna 132, at the first transition 140, at the second transition 146 and/or at the height of the fourth frontal radius diameter P4.

Sagittal radius diameters P9 to P12 of radius 134 and sagittal thicknesses of cortex P13 to P16 are preferably determined in an analogous manner from the bone structure in the sagittal plane (FIG. 12). The sagittal radius diameters P9 to P12 are preferably determined perpendicular to the frontal radius diameters P1 to P4. It is also preferred that the sagittal thicknesses of the cortex P13 to P16 are determined perpendicular to the frontal thicknesses of the cortex P5 to P8. In addition, the ulnar variance UV is determined as parameter P17 (not shown in FIG. 11).

A width P18 of the proximal carpal row 150 in the frontal plane E1 and/or a width P19 of the proximal carpal row 150 in the sagittal plane E2 are preferably determined as parameters. The width P18 is preferably measured in a lateral-medial direction R1 from a most lateral end 152 of the proximal carpal row 150 to a most medial end 154 of the proximal carpal row 150. The width P19 is preferably measured in an anterior-posterior direction R2 from a most anterior end 156 of the proximal carpal row 150 to a most posterior end 158 of the proximal carpal row 150. A height P20 of the proximal carpal row, measured between a most distal end 160 of the proximal carpal row and a most proximal end 162 of the proximal carpal row, is preferably a further parameter. A height of the carpal P21 is preferably determined between the most proximal end 162 and a most distal end 164 of a distal carpal row 166.

The angles β and ε are preferably determined and, in the present embodiment example, referred to as parameters P22 and parameters P23.

A frontal joint line P24 preferably bisects a distance between the proximal carpal row 150 and the joint surfaces 168, 170 of the radius 134 and the ulna 132. A sagittal joint line P25 preferably bisects a distance between the proximal carpal row 150 and the joint surface 168 of the radius 134. The frontal joint line P24 and/or the sagittal joint line P25 is preferably processed by an elliptical fitting and/or a circular fitting. A real course of the joint line is approximated by one or more circles and/or one or more ellipses.

In the present embodiment example, the position of a longitudinal axis of the radius 134 in extension through a third metacarpal bone 172 is determined as the final parameter P26.

The non-individualized 3D model of a wrist endoprosthesis 2 provided in step S1 is then individualized in step S4 based on the parameters P1 to P24. A diameter of the shaft 10 of the radius component 4 in the region of the connecting segment 40 is individualized here based on the parameters P1, P5, P9 and P12. A diameter of the shaft 10 at the connecting segment 40 in the frontal plane is preferably individualized such that said diameter corresponds to the difference between the frontal radius diameter P1 and the thickness of the cortex P5. In order to ensure that the shaft 10 is securely jammed in the radius 134, a jamming allowance, which is determined individually for each patient, is preferably added.

The jamming allowance preferably has a range from 0% to 25%, particularly preferably 5% to 10%, of the frontal radius diameter P1. The jamming allowance is calculated based on the cavity. In the case of a single radius, an increase in the radius, in the case of an elliptical shape, correspondingly along the axes of the ellipse.

P13 is subtracted from P9 and a jamming allowance is added in an analogous way. The allowance factor is preferably added symmetrically. Here, a cross section of the shaft 10 in the region of the connecting segment 40 is circular if a difference between P1 and P5 and P13 and P9 coincide, and elliptical if the differences between P1 and P5 and P13 and P9 differ. A diameter of the shaft 10 of the radius component 4 at the first transition 140, at the second transition 146 and in the region of the proximal shaft end 38 are preferably determined analogously from the parameters P2 to P4, P6 to P8, P10 to P12 and P14 to P16. The lateral surface 70 of the shaft 10 between the connecting segment 40 and the first transition 140 to the metaphysis 144 preferably comprises a porous surface structure. Growing in of the shaft 10 into the radius 134 can be improved by a porous surface structure. The lateral surface 70 of the shaft 10 is preferably smooth and/or polished between the first transition 140 and the proximal shaft end 38. The insertion of the shaft 10 into the radius 134 can thereby be facilitated in the course of treating a patient. The first joint surface 16 of the shaft 10 of the wrist endoprosthesis 2 is preferably aligned based on the parameters P22 and P23. The thickness D1 of the head 12 on the radius face 44 and the thickness D2 of the head 12 on the ulnar face 46 are particularly preferably adapted such that an ulnar variance UV disappears after an implantation of the radius component 4. It is also preferred that the ulnar variance UV of a natural wrist and the ulnar variance UV of a wrist fitted with a wrist endoprosthesis coincide. Furthermore, a shape of the first joint surface 16 is preferably adapted based on the frontal joint line P24 and/or sagittal joint line P25.

According to the present embodiment example, the second maximum width B2 of the carpal component 6 is individualized based on the width P18 of the proximal carpal row 150. The clear width W1 at the distal end 106 of the carpal cavity 18 and the maximum clear width W2 are individualized based on the width P19 of the proximal carpal row 150 in the sagittal plane E2.

The length L2 of the carpal component 6 is preferably individualized in step S4 based on the height of the wrist P21. A height P20 of the proximal carpal row 150 can also serve as a supplementary and/or stand-alone basis for the individualization of the length L2. Furthermore, the height P20 of the proximal carpal row 150 can preferably be used as a basis for individualizing the depth T1 of the carpal component 6, the depth T2 of the bulge 112 and/or the thickness D8 at the proximal end 108 of the carpal component 6. An allowance for individual adaptation in a range of 0% to 25%, particularly preferably 5% to 10%, of the height P20 and/or the height of the carpal P21 (carpal height) is preferably added.

In the present embodiment example, the second joint surface 24 is adapted based on the frontal joint line P24 and the sagittal joint line P25. The radius component 4 and the carpal component 6 are then aligned with one another, wherein the radius component 4 and/or the carpal component 6 are preferably disposed perpendicular to the determined position P26 of the longitudinal axis through the radius 134 and the third metacarpal bone 172.

The wrist endoprosthesis 2 is then produced in the fifth step S5.

FIGS. 13 a to c illustrate an individualized 3D model 174 of a carpal component 6 or an adapted anatomy-facing face 176 of the individualized 3D model 174 of a carpal component 6 of a wrist endoprosthesis 2. An outer face 178 of the 3D model 174 is implemented oval in a view from distal to proximal (FIG. 13a). Irritation of the tissue surrounding the later wrist endoprosthesis 2 can be prevented in this way. The anatomy-facing face 176 of the individualized 3D model 174, which here is the inner surface 98 of the carpal component 6, has been adapted to the patient-specific anatomy. The anatomy-facing face 176 comprises a plurality of elevations 178a, 178b, 178c which correspond to recesses of ligamentous connections of the wrist to be treated (not shown). A first elevation 178a corresponds here with a ligamentous connection between the triangular bone and the lunar bone. A second elevation 178b corresponds to a ligamentous connection between the lunar bone and the navicular bone. A third elevation corresponds to a ligamentous connection between the navicular bone and the small polygonal bone. FIG. 13b illustrates a view of the individualized 3D model 174 in the radial-ulnar direction. The outer face 178 is substantially U-shaped in the present view. Section lines of the anatomy-facing face 176, also illustrated by lines 180a, 180b, are substantially U-shaped. A height of the elevation 178a is greater than a height of the elevation 178b, so that a depth of the U-shape formed by line 180a is less than a depth of the U-shape formed by line 180b. It should be understood that preferably the entire anatomy-facing face 174 represents a negative shape 182 of the anatomy, particularly of the bony structure, of the wrist to be treated. In addition to elevations 178a, 178b, 178c, the remaining inner surface 98 is also adapted to the anatomy of the wrist to be treated (see FIGS. 13a, 13b). The elevations 178a, 178b, 178c can engage in corresponding recesses between the carpal bones and prevent the implanted wrist endoprosthesis 2 from slipping.

1. A wrist endoprosthesis (2) for functional replacement of the human wrist, comprising:
a radius component (4) that has
a shaft (10) for anchoring in the radius,
a head (12), and
a first joint surface (16),
which is formed on a distal head face (14), and
a carpal component (6) that has
a proximal carpal face (22),
a distal carpal face (20) and
a second joint surface (24),
which is formed on the proximal carpal face (22) and interacts with the first joint surface (16) of the radius component (4),
wherein
the carpal component (6) is substantially trough-shaped in order to at least partially surround the carpal bones.

2. The wrist endoprosthesis (2) according to claim 1, wherein the carpal component (6) comprises a carpal cavity (18) which is open to the distal carpal face (20).

3. The wrist endoprosthesis (2) according to claim 2, wherein the carpal cavity (18) has a maximum clear width (W2) measured in the sagittal plane (E2), wherein a corresponding clear width (W1) of the carpal cavity (18) at a distal end (106) the carpal component (6) is less than the maximum clear width (W2).

4. The wrist endoprosthesis (2) according to claim 1, wherein the carpal component (6) comprises one or two lateral openings (28, 32) which are formed in a face (26, 30) parallel to the sagittal plane (E2).

5. The wrist endoprosthesis according to claim 1, wherein the carpal component (6) tapers parallel to a frontal plane (E1) in the direction of the distal carpal face (20).

6. The wrist endoprosthesis (2) according to claim 1, wherein a proximal end (108) of the carpal component (6) is implemented as a thickening (110).

7. The wrist endoprosthesis (2) according to claim 1, wherein the wrist endoprosthesis (2) comprises luxation protection (8).

8. The wrist endoprosthesis (2) according to claim 7, wherein the luxation protection (8) comprises a band (34) which connects the carpal component (6) and the radius component (4).

9. The wrist endoprosthesis (2) according to claim 8, wherein the radius component (4) comprises a tunnel (36) extending from a dorsal face (64) of the radius component (4) to a palmar face (66) of the radius component (4), wherein the band (34) runs through the tunnel (36).

10. The wrist endoprosthesis (2) according to claim 1, wherein a first material forming the wrist endoprosthesis (2) is an isoelastic material, preferably a thermoplastic material, particularly preferably PEEK.

11. A wrist endoprosthesis (2) for partial functional replacement of the human wrist, comprising:
a carpal component (6) that has
a proximal carpal face (22),
a distal carpal face (20) and
a second joint surface (24),
which is disposed on the proximal carpal face (22) and formed to interact with a distal joint surface of a human radius,
wherein
the carpal component (6) is substantially trough-shaped in order to at least partially surround the carpal bones.

12. The wrist endoprosthesis (2) according to claim 11, wherein the proximal carpal face comprises a thickening (110) which is implemented as a convex elevation.

13. A method for producing a wrist endoprosthesis (2), particularly a wrist prosthesis (2) according to claim 1, the method comprising the steps:
a) providing or producing a non-individualized 3D model of a wrist endoprosthesis;
b) acquiring data material, particularly x-ray images, magnetic resonance tomography data, ultrasound images and/or computed tomography data, of a wrist of a patient to be treated;
c) determining one or more parameters from the data material for approximating an approximated joint structure of the patient from the data material;
d) individualizing the non-individualized 3D model based on the derived parameters for obtaining an individualized 3D model; and
e) producing the wrist endoprosthesis (2) by means of an additive manufacturing process based on the individualized 3D model.

14. The method according to claim 13, wherein the parameter represents at least one geometric shape of a recess of a ligamentous connection between adjacent carpal bones of the proximal carpal row of the wrist to be treated, and
wherein the individualized 3-D model (174) comprises an elevation (178a, 178b, 178c) corresponding to the recess of the ligamentous connection on a face (176) facing the anatomy.

15. The method according to claim 14, further comprising:
providing a surface structuring on at least one surface segment of the anatomy-facing face (176) of the individualized 3D model (174), wherein the production of the wrist endoprosthesis (2) takes place based on the individualized 3D model with surface structuring.

16. The method according to claim 15, wherein a second parameter represents a shape and position of a cartilage-covered joint surface of the wrist to be treated, a third parameter represents a shape and position of a cartilage-free surface of the wrist to be treated, and wherein the individualized 3D model (174) on the anatomy-facing face (176) comprises a first connecting surface corresponding to the cartilage-covered joint surface and a second connecting surface corresponding to the cartilage-free surface.

17. The method according to claim 16, wherein the surface structuring comprises first structural elements on the first connecting surface at least in segments and second structural elements on the second connecting surface at least in segments, which second structural elements are different from the first structural elements.

18. The method according to claim 17, wherein the first structural elements are structural elements protruding from the first connecting surface.

19. The method according to claim 18, wherein the first structural elements are rhombuses, rectangles, triangles, circular structures, ellipses, polygons and/or ribs.

20. The method according to claim 17, wherein the second structural elements are pores in the second connecting surface.

21. The method according to claim 13, wherein the wrist endoprosthesis (2) allows an independent and/or externally controlled unfolding.

22. The method according to claim 13, wherein in the event that step b) is not possible, at least one of the following steps is carried out:
acquiring data material, particularly x-ray images, magnetic resonance tomography data, ultrasound images and/or computed tomography data, of the second wrist of a patient; and/or
providing data from a statistical comparison group.

23. The method according to claim 13, comprising:
providing or creating a 3D instrument model of one or more surgical instruments based on the individualized 3D model of the wrist endoprosthesis and/or the data material; and
producing the surgical instrument based on the 3D instrument model.

24. The method according to claim 13, wherein a first material used for producing the wrist endoprosthesis (2) is an isoelastic material, preferably a plastic, particularly preferably PEEK.

25. A method for producing a wrist endoprosthesis (2), particularly a wrist prosthesis (2) according to claim 1, wherein the method comprises the steps:
a) providing or producing a non-individualized 3D model of a wrist endoprosthesis;
b) acquiring data material, particularly x-ray images, magnetic resonance tomography data, ultrasound images and/or computed tomography data, of a wrist of a patient to be treated;
c) creating a patient-specific 3D model of at least one anatomy segment of the wrist to be treated using the data material;
d) deriving a negative shape (182) of the anatomy segment of the wrist to be treated;
e) adapting an anatomy-facing face of the non-individualized 3D model to the derived negative shape (182) of the anatomy segment of the wrist to be treated for obtaining an individualized 3D model; and
f) producing the wrist endoprosthesis (2) by means of an additive manufacturing process based on the individualized 3D model.

26. A computer program product comprising code means which, when executed on a computer, are implemented to execute at least one of the steps of the method defined in claim 13.
